(19) United States
(12) Patent Application Publication
(10) Pub. No.: US 2008/0097108 A1
(43) Pub. Date: Apr. 24, 2008

(54) Ophthalmic Compositions for Treating Ocular Hypertension

(76) Inventors: Ying-Duo Gao, Edison, NJ (US); Dong-Ming Shen, Edison, NJ (US)

Correspondence Address: MERCK AND CO., INC, P O BOX 2000, RAHWAY, NJ, 07065-0907, US

(21) Appl. No.: 11/661,649

(22) PCT Filed: Oct. 7, 2005
(86) PCT No.: PCT/US05/36039

§ 371 Date: Feb. 27, 2007

Related U.S. Application Data
(60) Provisional application No. 60/618,541, filed on Oct. 13, 2004.

(51) Int. Cl. C07D 231/54

(52) U.S. Cl. 548/359.1

(57) ABSTRACT

This invention relates to potent potassium channel blocker compounds of Formula (I) or a formulation thereof for the treatment of glaucoma and other conditions which leads to elevated intraoccular pressure in the eye of a patient. This invention also relates to the use of such compounds to provide a neuroprotective effect to the eye of mammalian species, particularly humans.

This case claims the benefit of U.S. Provisional application 60/618,541, filed Oct. 13, 2004.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

There are several therapies for treating glaucoma and elevated intraocular pressure, but the efficacy and the side effect profiles of these agents are not ideal. Recently potassium channel blockers were found to reduce intraocular pressure in the eye and therefore provide yet one more approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Opthalmol. Vis. Sci 38, 1997; WO 89/10757, WO94/28900, and WO 96/33719).

### SUMMARY OF THE INVENTION

This invention relates to the use of potent potassium channel blockers or a formulation thereof in the treatment of glaucoma and other conditions which are related to elevated intraocular pressure in the eye of a patient. This invention also relates to the use of such compounds to provide a neuroprotective effect to the eye of mammalian species, particularly humans. More particularly this invention relates to the treatment of glaucoma and/or ocular hypertension (elevated intraocular pressure) using novel tetrahydrocarbazoles and related compounds having structural formula I: 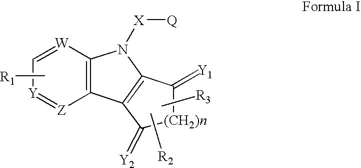
or a pharmaceutically acceptable salt, in vivo hydrolysable ester, enantiomer, diastereomer, geometric isomers or mixture thereof:
wherein,
Y₁ and Y₂ are independently O; H₂; H and R₃; H and R₂; or R₂ and R₃;
X represents —(CHR₇)ₚ—, or —(CHR₇)ₚCO—;
W, Y and Z independently are CH and N;
R₁ represents hydrogen, C₁₋₆ alkoxy, OH, C₃₋₈ cycloalkoxy, C₁₋₆ alkyl, C₃₋₈ cycloalkyl, C₁₋₆ alkenyl, S(O)_{q}R, COOR, COR, SO₃H, —O(CH₂)ₙN(R)₂, —O(CH₂)ₙCO₂R, —OPO(OH)₂, C₆₋₁₀ aryl, C₅₋₁₀ heteroaryl, C₅₋₁₀ heterocyclyl, CF₃, OCF₃, N(R)₂, nitro, cyano, C₁₋₆ alkylamino, or halogen, said aryl, alkyl, alkoxy, heterocyclyl, aryl or heteroaryl optionally substituted with 1-3 groups of R^{a};
R₂ and R₃ independently represent hydrogen, C₁₋₆ alkoxy, OH, C₁₋₆ alkyl, C₁₋₆ alkyl-S, C₁₋₆ alkyl-CO—, C₁₋₆ alkenyl, C₃₋₈ cycloalkoxy, C₃₋₈ cycloalkyl, C₃₋₈ cycloalkyl-S, C₃₋₈ cycloalkyl-CO—, COOR, SO₃H, —O(CH₂)ₙN(R)₂, —O(CH₂)ₙCO₂R, —OPO(OH)₂, C₆₋₁₀ aryl, C₅₋₁₀ heteroaryl, C₅₋₁₀ heterocyclyl, CF₃, —N(R)₂, nitro, cyano, C₁₋₆ alkylamino, or halogen, said aryl, alkyl, alkoxy, heterocyclyl, aryl or heteroaryl optionally substituted with 1-3 groups of R^{a};
or R₂ and R₃ can join together with the intervening atoms in the ring to form a 4-8 membered ring, This ring can be interrupted with 1-2 atoms chosen from N, O, and S and/or having 1-4 double bonds.
Q represents hydrogen, C₁₋₁₀ alkyl, —(CH₂)ₙ(CHR)_{q}(CH₂)ₘOR₉, —(CH₂)ₙ(CHR)_{q}(CH₂)ₘNR₈R₉, —(CH₂)ₙ(CHR)_{q}(CH₂)ₘC₃₋₈ cycloalkyl, —(CH₂)ₙ(CHR)_{q}(CH₂)ₘC₅₋₁₄ fused cycloalkyl, —(CH₂)ₙ(CHR)_{q}(CH₂)ₘC₃₋₁₀ heterocyclyl, —(CH₂)ₙ(CHR)_{q}(CH₂)ₘC₅₋₁₀ heteroaryl, —(CH₂)ₙ(CHR)_{q}(CH₂)ₘCOOR, —(CH₂)ₙ(CHR)_{q}(CH₂)ₘC₆₋₁₀ aryl, —(CH₂)ₙ(CHR)_{q}(CH₂)ₘNHR₉, —(CH₂)ₙ(CHR)_{q}(CH₂)ₘNHCOOR, —(CH₂)ₙ(CHR)_{q}(CH₂)ₘN(R₉)CO₂R, —(CH₂)ₙ(CHR)_{q}(CH₂)ₘN(R₉)COR, —(CH₂)ₙ(CHR)_{q}(CH₂)ₘNHCOR, —(CH₂)ₙ(CHR)_{q}(CH₂)ₘCONH(R₉), aryl, CF₃, (CH₂)ₙ(CHR)_{q}(CH₂)ₘSO₂R, —(CH₂)ₙ(CHR)_{q}(CH₂)ₘSO₂N(R)₂, —(CH₂)ₙCON(R)₂, —(CH₂)ₙCONHC(R)₃, —(CH₂)ₙCONHC(R)₂CO₂R, —(CH₂)ₙCOR₉, nitro, cyano or halogen, said alkyl, alkoxy, heterocyclyl, aryl or heteroaryl optionally substituted with 1-3 groups of R^{a};
R represents hydrogen, or C₁₋₆ alkyl, C₃₋₈ cycloalkyl, C₆₋₁₀ aryl, or C₅₋₁₀ heteroaryl;
R₇ represents hydrogen, C₁₋₆ alkyl, —(CH₂)ₙCOOR, —(CH₂)ₙCOR or —(CH₂)ₙN(R)₂,
R₈ represents hydrogen, C₁₋₁₀ alkyl, C₂₋₆ alkenyl, C₁₋₆ alkylSR, —(CH₂)ₙO—(CH₂)ₘOR, —(CH₂)ₙ(CHR)_{q}(CH₂)ₘC₁₋₆ alkoxy, —(CH₂)ₙ(CHR)_{q}(CH₂)ₘC₃₋₈ cycloalkyl, —(CH₂)ₙ(CHR)_{q}(CH₂)ₘC₃₋₁₀ heterocyclyl, —N(R)₂, —(CH₂)ₙ(CHR)_{q}(CH₂)ₘCOOR, or —(CH₂)ₙ(CHR)_{q}(CH₂)ₘC₆₋₁₀ aryl, —(CH₂)ₙ(CHR)_{q}(CH₂)ₘC₅₋₁₀ heteroaryl, said alkyl, alkenyl, alkoxy, heterocyclyl, or aryl optionally substituted with 1-3 groups selected from R^{a};
R₉ represents hydrogen, C₁₋₁₀ alkyl, C₁₋₆ alkylSR, —(CH₂)ₙO—(CH₂)ₘOR, —(CH₂)ₙ(CHR)_{q}(CH₂)ₘC₁₋₆ alkoxy, —(CH₂)ₙ(CHR)_{q}(CH₂)ₘC₃₋₈ cycloalkyl, —(CH₂)ₙ(CHR)_{q}(CH₂)ₘC₃₋₁₀ heterocyclyl, —(CH₂)ₙ(CHR)_{q}(CH₂)ₘC₅₋₁₀ heteroaryl, —(CH₂)ₙ(CHR)_{q}(CH₂)ₘN(R)₂, CH₂)ₙ(CHR)_{q}(CH₂)ₘNHR, —(CH₂)ₙ(CHR)_{q}(CH₂)ₘCOOR, or (CH₂)ₙ(CHR)_{q}(CH₂)ₘC₆₋₁₀ aryl, —(CH₂)ₙ(CHR)_{q}(CH₂)ₘNRCOOR, —(CH₂)ₙ(CHR)_{q}(CH₂)ₘCOR, (CH₂)ₙ(CHR)_{q}(CH₂)ₘSO₂R, —(CH₂)ₙ(CHR)_{q}(CH₂)ₘSO₂N(R)₂, said alkyl, alkoxy, cycloalkyl, heterocyclyl, aryl or heteroaryl optionally substituted with 1-3 groups selected from R^{a};
or, R₈ and R₉ taken together with the intervening “N” of NR₈R₉ of Q form a 3-10 membered carbocyclic or heterocyclic—
ring optionally interrupted by 1-2 atoms of O, S, C(O) or NR, and optionally having 1-4 double bonds, and optionally substituted by 1-3 groups selected from R^{a};
R^{a} represents F, Cl, Br, 1, CF₃, OH, N(R)₂, NO₂, CN, —COR, —CONHR, —CONR₂, —O(CH₂)ₙCOOR, —NH(CH₂)ₙOR, —COOR, —OCF₃, —NHCOR, —SO₂R, —SO₂NR, —SR, (C₁-C₆ alkyl)O—, —(CH₂)ₙO—(CH₂)ₘOR, —(CH₂)ₙC₁₋₆ alkoxy, (aryl)O—, —(CH₂)ₙOH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R^{w}, —(C₁-C₆ alkyl)-C₃₋₁₀ heterocyclyl-R_{w}, —(CH₂)ₙ-Z¹-C(=Z²)N(R)₂, —(C₂₋₆ alkenyl)NR^{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆ alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-Z¹-C(=Z²)N(R)₂, —(CH₂)ₙSO₂R, —(CH₂)ₙSO₃H, —(CH₂)ₙPO(OR)₂, —(CH₂)ₙOPO(OR)₂, C₃₋₁₀cycloalkyl, C₆₋₁₀ aryl, C₃₋₁₀ heterocyclyl, C₂₋₆ alkenyl, and C₁-C₁₀ alkyl, said alkyl, alkenyl, alkoxy, heterocyclyl and aryl optionally substituted with 1-3 groups selected from C₁-C₆ alkyl, CN, NO₂, OH, CON(R)₂ and COOR;
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
Z¹ and Z² independently represents NR_{w}, O, CH₂, or S;
m is 0-3;
n is 0-4;
p is 0-1; and
q is 0-2.

This and other aspects of the invention will be realized upon inspection of the invention as a whole.

### DETAILED DESCRIPTION OF THE INVENTION

The present invention is directed to novel potassium channel blockers of Formula I. It also relates to a method for decreasing elevated intraocular pressure or treating glaucoma by administration, preferably topical or intra-camaral administration, of a composition containing a potassium channel blocker of Formula I described hereinabove and a pharmaceutically acceptable carrier.

One embodiment of this invention is realized when Q is C₁₋₁₀ alkyl, —(C₁₋₆-alkyl)ₙOR, or (CH₂)ₙ(CHR)_{q}(CH₂)ₘNR₈R₉ and all other variables are as originally described.

Another embodiment of this invention is realized when W═Y=Z=CH and all other variables are as originally described.

Another embodiment of this invention is realized when R₁ is C₁₋₆ alkoxy, OH, C₁₋₆ alkyl and all other variables are as originally described.

Another embodiment of this invention is realized when X is —(CHR₇)ₚ— and all other variables are as originally described.

Another embodiment of this invention is realized when X is —(CHR₇)ₚCO— and all other variables are as originally described.

Still another embodiment of this invention is realized when Y₁ and Y₂ are both H₂, or one of Y₁ and Y₂ is O and the other is H₂ and all other variables are as originally described.

Still another embodiment of this invention is realized when one of Y₁ and Y₂ is H and R₃ and the other is H and R₂ and all other variables are as originally described.

Another embodiment of this invention is realized when Q is C₁₋₁₀ alkyl, or (CH₂)ₙ(CHR)_{q}(CH₂)ₘNR₈R₉; X is —(CHR₇)ₚCO—; R₃ and R₂ independently are H and C₁₋₆ alkyl; and Y₁ and Y₂ are H₂ and all other variables are as originally described.

Another embodiment of the instant invention is realized when R^{a} is selected from F, Cl, Br, I, OH, CF₃, N(R)₂, NO₂, CN, —CONHR, —CONR₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙO—(CH₂)ₘOR, —(CH₂)ₙC₁₋₆ alkoxy, (aryl)O—, —(CH₂)ₙOH, (C₁-C₆ alkyl)S(O)ₘ—, H₂N—C(NH)—, (C₁-C₆ alkyl)C(O)—, —(CH₂)ₙPO(OR)₂, —(CH₂)ₙOPO(OR)₂, C₂₋₆ alkenyl, and C₁-C₁₀ alkyl, said alkyl and alkenyl, optionally substituted with 1-3 groups selected from C₁-C₆ alkyl, and COOR;

Examples of compounds of formula I of this invention are:
1-(7-methoxy-1,2,3,4-tetrahydro-9H-carbazol-9-yl)-3,3-dimethylbutan-2-one,
N,N-dibutyl-2-(7-methoxy-1,2,3,4-tetrahydro-9H-carbazol-9-yl)acetamide,
2-(7-methoxy-1,2,3,4-tetrahydro-9H-carbazol-9-yl)-N,N-dipropylacetamide,
2-(7-methoxy-1,2,3,4-tetrahydro-9H-carbazol-9-yl)-N,N-bis(3-methylbutyl)acetamide,
N-ethyl-2-(7-methoxy-1,2,3,4-tetrahydro-9H-carbazol-9-yl)-N-(3-methylbutyl)acetamide,
N,N-diisobutyl-2-(7-methoxy-1,2,3,4-tetrahydro-9H-carbazol-9-yl)acetamide,
N-(cyclopropylmethyl)-2-(7-methoxy-1,2,3,4-tetrahydro-9H-carbazol-9-yl)-N-propylacetamide,
N-cyclohexyl-N-ethyl-2-(7-methoxy-1,2,3,4-tetrahydro-9H-carbazol-9-yl)acetamide,
N-butyl-N-ethyl-2-(7-methoxy-1,2,3,4-tetrahydro-9H-carbazol-9-yl)acetamide,
N-butyl-2-(7-methoxy-1,2,3,4-tetrahydro-9H-carbazol-9-yl)-N-propylacetamide,
7-methoxy-9-[2-(trans-octahydroisoquinolin-2(1H)-yl)-2-oxoethyl]-2,3,4,9-tetrahydro-1H-carbazole,
7-methoxy-9-[2-(cis-octahydroisoquinolin-2(1H)-yl)-2-oxoethyl]-2,3,4,9-tetrahydro-1H-carbazole,
9-[2-(trans-2,5-dipropylpyrrolidin-1-yl)-2-oxoethyl]-7-methoxy-2,3,4,9-tetrahydro-1H-carbazole,
9-[2-(cis-2,5-dipropylpyrrolidin-1-yl)-2-oxoethyl]-7-methoxy-2,3,4,9-tetrahydro-1H-carbazole,
N-(3,3-dimethylbutyl)-N-ethyl-2-(7-methoxy-1,2,3,4-tetrahydro-9H-carbazol-9-yl)acetamide,
N-ethyl-2-(7-methoxy-1,2,3,4-tetrahydro-9H-carbazol-9-yl)-N-1,3-thiazol-2-ylacetamide,
N-(2,2-dimethylpropyl)-N-ethyl-2-(7-methoxy-1,2,3,4-tetrahydro-9H-carbazol-9-yl)acetamide 1-(5-methoxy-1,2,3,4-tetrahydro-9H-carbazol-9-yl)-3,3-dimethylbutan-2-one,
N,N-dibutyl-2-(5-methoxy-1,2,3,4-tetrahydro-9H-carbazol-9-yl)acetamide,
2-(5-methoxy-1,2,3,4-tetrahydro-9H-carbazol-9-yl)-N,N-dipropylacetamide,
N-ethyl-2-(5-methoxy-1,2,3,4-tetrahydro-9H-carbazol-9-yl)-N-(3-methylbutyl)acetamide,
1-(7-methoxy-2,2-dimethyl-1,2,3,4-tetrahydro-9H-carbazol-9-yl)-3,3-dimethylbutan-2-one,
4-(7-methoxy-2,2-dimethyl-1,2,3,4-tetrahydro-9H-carbazol-9-yl)-2,2,7,7-tetramethyloctane-3,6-dione,
9-(3,3-dimethylbutyl)-7-methoxy-2,2-dimethyl-2,3,4,9-tetrahydro-1H-carbazole,
2-(7-methoxy-2,2-dimethyl-1,2,3,4-tetrahydro-9H-carbazol-9-yl)-N,N-dipropylacetamide,
2-(7-methoxy-2,2-dimethyl-1,2,3,4-tetrahydro-9H-carbazol-9-yl)-N,N-bis(3-methylbutyl)acetamide,
N-ethyl-2-(7-methoxy-2,2-dimethyl-1,2,3,4-tetrahydro-9H-carbazol-9-yl)-N-(3-methylbutyl)acetamide,
N,N-diisobutyl-2-(7-methoxy-2,2-dimethyl-1,2,3,4-tetrahydro-9H-carbazol-9-yl)acetamide,
N-(cyclopropylmethyl)-2-(7-methoxy-2,2-dimethyl-1,2,3,4-tetrahydro-9H-carbazol-9-yl)-N-propylacetamide,
N-cyclohexyl-N-ethyl-2-(7-methoxy-2,2-dimethyl-1,2,3,4-tetrahydro-9H-carbazol-9-yl)acetamide,
N-butyl-N-ethyl-2-(7-methoxy-2,2-dimethyl-1,2,3,4-tetrahydro-9H-carbazol-9-yl)acetamide,
N-butyl-2-(7-methoxy-2,2-dimethyl-1,2,3,4-tetrahydro-9H-carbazol-9-yl)-N-propylacetamide,
7-methoxy-2,2-dimethyl-9-[2-(trans-octahydroisoquinolin-2(1H)-yl)-2-oxoethyl]-2,3,4,9-tetrahydro-1H-carbazole,
7-methoxy-2,2-dimethyl-9-[2-(cis-octahydroisoquinolin-2(1H)-yl)-2-oxoethyl]-2,3,4,9-tetrahydro-1H-carbazole,
9-[2-(trans-2,5-dipropylpyrrolidin-1-yl)-2-oxoethyl]-7-methoxy-2,2-dimethyl-2,3,4,9-tetrahydro-1H-carbazole,
9-[2-(cis-2,5-dipropylpyrrolidin-1-yl)-2-oxoethyl]-7-methoxy-2,2-dimethyl-2,3,4,9-tetrahydro-1H-carbazole,
N-(3,3-dimethylbutyl)-N-ethyl-2-(7-methoxy-2,2-dimethyl-1,2,3,4-tetrahydro-9H-carbazol-9-yl)acetamide,
N-ethyl-2-(7-methoxy-2,2-dimethyl-1,2,3,4-tetrahydro-9H-carbazol-9-yl)-N-1,3-thiazol-2-ylacetamide
N-(3,3-dimethylbutyl)-2-(7-methoxy-2,2-dimethyl-1,2,3,4-tetrahydro-9H-carbazol-9-yl)-N-propylacetamide,
9-(3,3-Dimethylbutyl)-7-methoxy-1,2,3,9-tetrahydro-4H-carbazol-4-one,
2-(7-methoxy-4-oxo-1,2,3,4-tetrahydro-9H-carbazol-9-yl)-N,N-bis(3-methylbutyl)acetamide,
N-ethyl-2-(7-methoxy-4-oxo-1,2,3,4-tetrahydro-9H-carbazol-9-yl)-N-(3-methylbutyl)acetamide,
N-butyl-2-(7-methoxy-4-oxo-1,2,3,4-tetrahydro-9H-carbazol-9-yl)-N-propylacetamide,
9-[2-(trans-2,5-dipropylpyrrolidin-1-yl)-2-oxoethyl]-7-methoxy-4-oxo-2,3,4,9-tetrahydro-1H-carbazole,
9-[2-(cis-2,5-dipropylpyrrolidin-1-yl)-2-oxoethyl]-7-methoxy-4-oxo-2,3,4,9-tetrahydro-1H-carbazole,
N-(3,3-dimethylbutyl)-N-ethyl-2-(7-methoxy-4-oxo-1,2,3,4-tetrahydro-9H-carbazol-9-yl)acetamide,
N-ethyl-2-(7-methoxy-4-oxo-1,2,3,4-tetrahydro-9H-carbazol-9-yl)-N-1,3-thiazol-2-ylacetamide,
N-(3,3-dimethylbutyl)-2-(7-methoxy-4-oxo-1,2,3,4-tetrahydro-9H-carbazol-9-yl)-N-propylacetamide,
   or a pharmaceutically acceptable salt, in vivo hydrolysable ester, enantiomer, diastereomer or mixture thereof.

The invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190)

When any variable (e.g. aryl, heterocycle, R¹, R⁶ etc.) occurs more than one time in any constituent, its definition on each occurrence is independent at every other occurrence.

Also, combinations of substituents/or variables are permissible only if such combinations result in stable compounds.

When R^{a} is —O— and attached to a carbon it is referred to as a carbonyl group and when it is attached to a nitrogen (e.g., nitrogen atom on a pyridyl group) or sulfur atom it is referred to an N-oxide and sulfoxide group, respectively.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopropyl cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, unless otherwise defined, without alternating or resonating double bonds between carbon atoms. It may contain from 1 to 4 rings, which can be fused. Examples of such cycloalkyl elements include, but are not limited to, cyclopropyl, cyclobutyl, cyclopentyl, cyclohexyl, cycloheptyl, adamantyl, diamantyl, [2.2.2]bicyclooctyl, and [1.1.1]bicyclopentyl.

Alkenyl is C₂-C₆ alkenyl.

Alkoxy refers to an alkyl group of indicated number of carbon atoms attached through an oxygen bridge, with the alkyl group optionally substituted as described herein. Said groups are those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

Halogen (halo) refers to chlorine, fluorine, iodine or bromine.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Examples of aryl groups are phenyl, naphthyl, tetrahydronaphthyl, indanyl, biphenyl, phenanthryl, anthryl or acenaphthyl and phenanthrenyl, preferably phenyl, naphthyl or phenanthrenyl. Aryl groups may likewise be substituted as defined. Preferred substituted aryls include phenyl and naphthyl.

The term heterocyclyl or heterocyclic, as used herein, represents a stable 3- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, dihydropyrrolyl, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. Preferably, heterocycle is selected from 2-azepinonyl, benzimidazolyl, 2-diazapinonyl, dihydroimidazolyl, dihydropyrrolyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl. Additional nitrogen atoms may be present together with the first nitrogen and oxygen or sulfur, giving, e.g., thiadiazole.

This invention is also concerned with compositions and methods of treating ocular hypertension or glaucoma by administering to a patient in need thereof one of the compounds of formula I alone or in combination with one or more of the following active ingredients, in combination with a adrenergic blocking agent such as timolol, betaxolol, levobetaxolol, carteolol, levobunolol, a parasympathomimetic agent such as epinephrine, iopidine, brimonidine, clonidine, para-aminoclonidine, carbonic anhydrase inhibitor such as dorzolamide, acetazolamide, metazolamide or brinzolamide, an EP4 agonist (such as those disclosed in WO 02/24647, WO 02/42268, EP 1114816, WO 01/46140, PCT Appln. No. CA2004000471, and WO 01/72268), a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine or a mixture thereof. An example of a hypotensive lipid (the carboxylic acid group on the α-chain link of the basic prostaglandin structure is replaced with electrochemically neutral substituents) is that in which the carboxylic acid group is replaced with a C₁₋₆ alkoxy group such as OCH₃ (PGF₂ₐ 1-OCH₃), or a hydroxy group (PGF₂ₐ 1-OH).

Preferred potassium channel blockers are calcium activated potassium channel blockers. More preferred potassium channel blockers are high conductance, calcium activated potassium (Maxi-K) channel blockers. Maxi-K channels are a family of ion channels that are prevalent in neuronal, smooth muscle and epithelial tissues and which are gated by membrane potential and intracellular Ca²⁺.

The present invention is based upon the finding that Maxi-K channels, if blocked, inhibit aqueous humor production by inhibiting net solute and H₂O efflux and therefore lower IOP. This finding suggests that Maxi-K channel blockers are useful for treating other ophthamological dysfunctions such as macular edema and macular degeneration. It is known that lowering IOP promotes blood flow to the retina and optic nerve. Accordingly, the compounds of this invention are useful for treating macular edema and/or macular degeneration.

It is believed that Maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof.

A number of marketed drugs function as potassium channel antagonists. The most important of these include the compounds Glyburide, Glipizide and Tolbutamide. These potassium channel antagonists are useful as antidiabetic agents. The compounds of this invention may be combined with one or more of these compounds to treat diabetes.

Potassium channel antagonists are also utilized as Class 3 antiarrhythmic agents and to treat acute infarctions in humans. A number of naturally occurring toxins are known to block potassium channels including Apamin, Iberiotoxin, Charybdotoxin, Noxiustoxin, Kaliotoxin, Dendrotoxin(s), mast cell degranuating (MCD) peptide, and β-Bungarotoxin (β-BTX). The compounds of this invention may be combined with one or more of these compounds to treat arrhythmias.

Depression is related to a decrease in neurotransmitter release. Current treatments of depression include blockers of neurotransmitter uptake, and inhibitors of enzymes involved in neurotransmitter degradation which act to prolong the lifetime of neurotransmitters.

Alzheimer's disease is also characterized by a diminished neurotransmitter release. Three classes of drugs are being investigated for the treatment of Alzheimer's disease cholinergic potentiators such as the anticholinesterase drugs (e.g., physostigmine (eserine), and Tacrine (tetrahydroaminocridine)); nootropics that affect neuron metabolism with little effect elsewhere (e.g., Piracetam, Oxiracetam; and those drugs that affect brain vasculature such as a mixture of ergoloid mesylates amd calcium channel blocking drugs including Nimodipine. Selegiline, a monoamine oxidase B inhibitor which increases brain dopamine and norepinephrine has reportedly caused mild improvement in some Alzheimer's patients. Aluminum chelating agents have been of interest to those who believe Alzheimer's disease is due to aluminum toxicity. Drugs that affect behavior, including neuroleptics, and anxiolytics have been employed. Anxiolytics, which are mild tranquilizers, are less effective than neuroleptics The present invention is related to novel compounds which are useful as potassium channel antagonists.

The compounds of this invention may be combined with anticholinesterase drugs such as physostigmine (eserine) and Tacrine (tetrahydroaminocridine), nootropics such as Piracetam, Oxiracetam, ergoloid mesylates, selective calcium channel blockers such as Nimodipine, or monoamine oxidase B inhibitors such as Selegiline, in the treatment of Alzheimer's disease. The compounds of this invention may also be combined with Apamin, Iberiotoxin, Charybdotoxin, Noxiustoxin, Kaliotoxin, Dendrotoxin(s), mast cell degranuating (MCD) peptide, β-Bungarotoxin (β-BTX) or a combination thereof in treating arrythmias. The compounds of this invention may further be combined with Glyburide, Glipizide, Tolbutamide or a combination thereof to treat diabetes.

The herein examples illustrate but do not limit the claimed invention. Each of the claimed compounds are potassium channel antagonists and are thus useful in the described neurological disorders in which it is desirable to maintain the cell in a depolarized state to achieve maximal neurotransmitter release. The compounds produced in the present invention are readily combined with suitable and known pharmaceutically acceptable excipients to produce compositions which may be administered to mammals, including humans, to achieve effective potassium channel blockage.

For use in medicine, the salts of the compounds of formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

As used herein, the term “composition” is intended to encompass a product comprising the specified ingredients in the specific amounts, as well as any product which results, directly or indirectly, from combination of the specific ingredients in the specified amounts.

When a compound according to this invention is administered into a human subject, the daily dosage will normally be determined by the prescribing physician with the dosage generally varying according to the age, weight, sex and response of the individual patient, as well as the severity of the patient's symptoms.

The maxi-K channel blockers used can be administered in a therapeutically effective amount intravenously, subcutaneously, topically, transdermally, parenterally or any other method known to those skilled in the art.

Ophthalmic pharmaceutical compositions are preferably adapted for topical administration to the eye in the form of solutions, suspensions, ointments, creams or as a solid insert. Ophthalmic formulations of this compound may contain from 0.01 ppm to 5% and especially 0.1 ppm to 1% of medicament. Higher dosages as, for example, about 10% or lower dosages can be employed provided the dose is effective in reducing intraocular pressure, treating glaucoma, increasing blood flow velocity or oxygen tension. For a single dose, from between 1 ng to 5000 μg, preferably 10 ng to 500 μg, and especially 100 ng to 200 μg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mammalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The following examples, given by way of illustration, are demonstrative of the present invention. Definitions of the terms used in the examples are as follows:

HOBt—1-hydroxybenzotriazole hydrate

EDC—1-(3-Dimethylaminopropyl)-3-ethylcarbodiimide hydrochloride

NMR—nuclear magnetic resonance,

TFA—trifluoroacetic acid,

DIEA—N,N-diisopropylethylamine

SGC—silica gel chromatography,

h=hr=hour,

THF—tetrahydrofuran,

DMF—dimethylformamide,

min—minute,

LC/MS—liquid chromatography/mass spectrometry,

RP-HPLC—reverse phase high performance liquid chromatography,

equiv=eq=equivalent,

General Experimental Conditions: NMR spectra were recorded at room temperature on Varian Instruments referenced to residual solvent peak. LC-MS were measured on an Aglient HPLC and Micromass ZQ detector with electrospray ionization using a 2.0×50 mm X-Terra C18 column and 10˜98% MeCN gradient over 3.75 minutes followed by 98% MeCN for 1 minute. The aqueous and MeCN eluents contained 0.06 and 0.05% (v/v) trifluoroacetic acid, respectively. Mass peaks are listed in decreasing order of relative abundance. Preparative HPLC separations were done using a C18 column such as YMC 20×150 mm 5μ ProC18, Phenomenex 100×21.2 mm 5μ C18 Luna, or a 9.4×250 mm SB-C18 Zorbax column.

The following examples given by way of illustration are demonstrative of the present invention. The compounds of this invention can be made, with modification where appropriate, in accordance with the Schemes below.

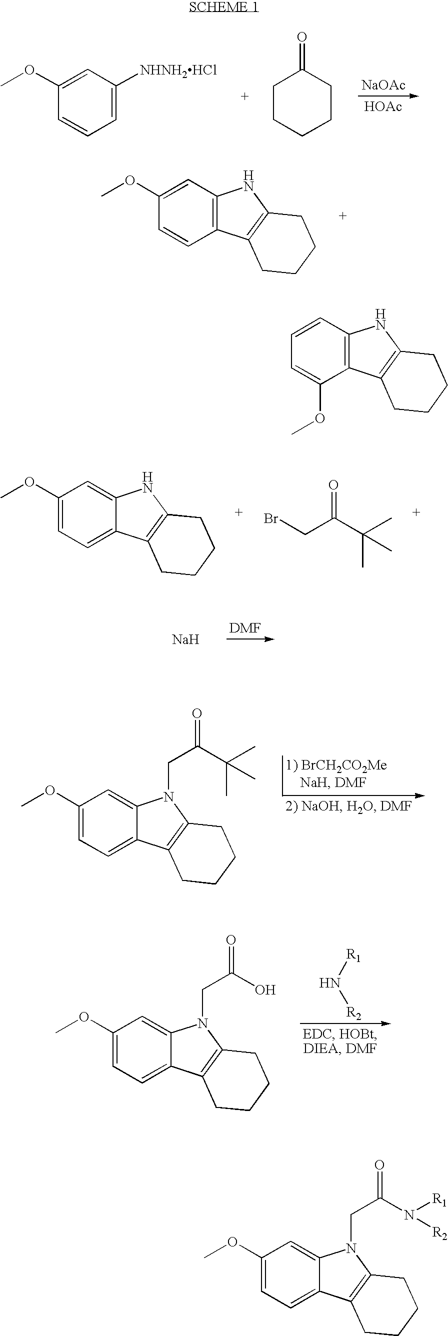

Scheme 1 shows the preparation of tetrahydrocarbazole class of potassium channel modifiers. Fisher indole synthesis using cyclohexanone and 3-methoxyphenyl hydrazine provided a mixture of two methoxy tetrahydrocarbazoles. They were separated by SGC. They can be alkylated by bromoketone to give the final product. Alkylation with α-bromoacetate, followed by hydrolysis to acid and amide formation, provided acetamide derivatives. An analogous method was used to prepare substituted tetrahydrocarbazoles as illustrated in Scheme 2.

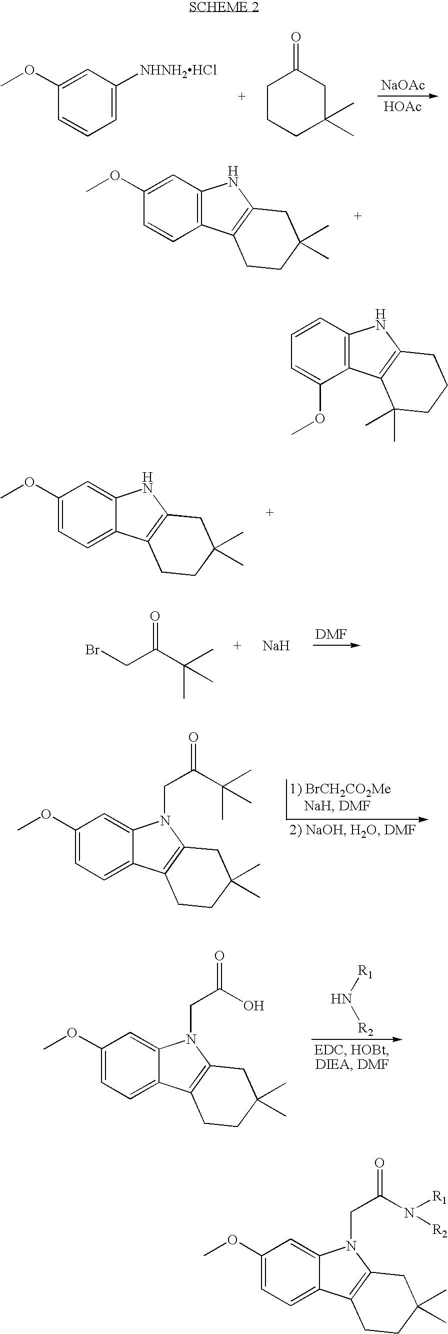

Several methods have been reported for the preparation of oxo-tetrahydrocarbazoles. For example, Scott et al. (Tetrahedron; 59; 33; 2003; 6323) and Iyer et al.; (J. Chem. Soc. Perkin Trans. 2; 1973; 878) had reported approaches to 7-methoxy-1,2,3,9-tetrahydro-4H-carbazol-4-one and 7-methoxy-2,3,4,9-tetrahydro-1H-carbazol-1-one, respectively. We used a modified method of Iida et al. (J. Org. Chem. 1980, 45, 2938) for the synthesis of 7-methoxy-1,2,3,9-tetrahydro-4H-carbazol-4-one, using copper (II) chloride instead of oxygen in the indole formation step (Scheme 3). The rest of the steps were similar to those used previously.

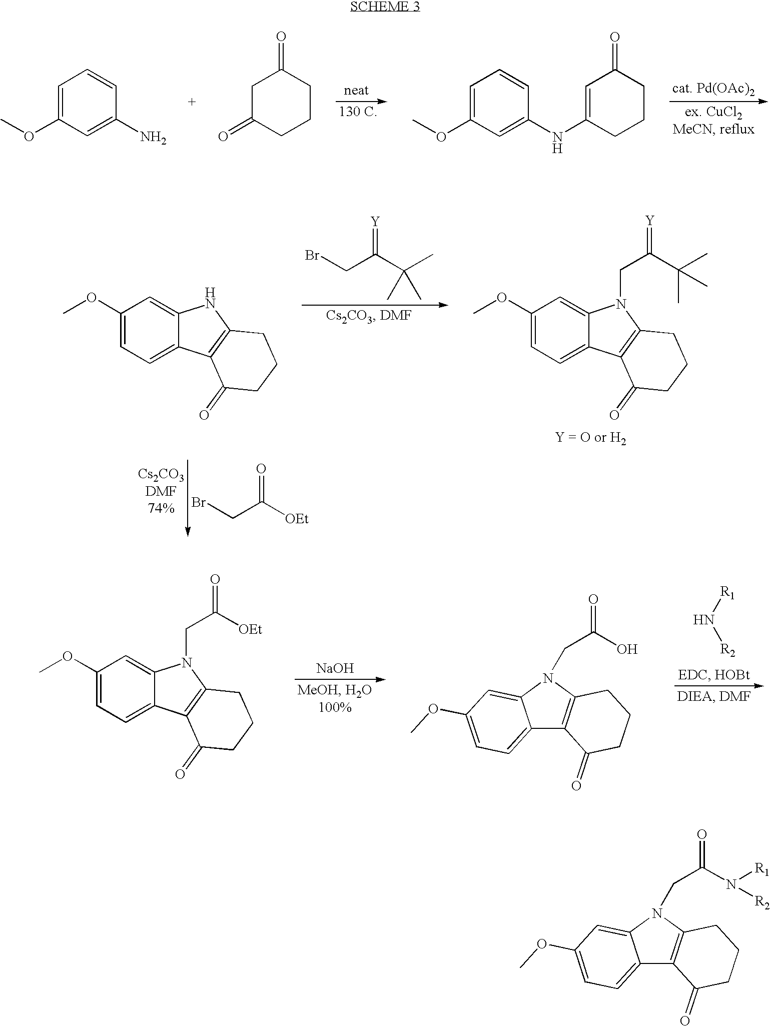

### EXAMPLE 1

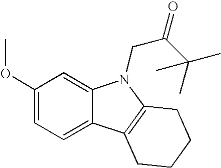

### 1-(7-Methoxy-1,2,3,4-tetrahydro-9H-carbazol-9-yl)-3,3-dimethylbutan-2-one

### Step A. 7-Methoxy-2,3,4,9-tetrahydro-1H-carbazole

A mixture of 4.04 g 3-methoxyphenylhydrazine hydrochloride, 2.27 g cyclohexanone, and 1.90 g sodium acetate in 16 mL acetic acid was refluxed under nitrogen for 4 hours. The solvents were removed under reduced pressure. The residue was partitioned between water and EtOAc. The combined EtOAc extract was Wash the combined organic layer with 0.1 N HCl, 5% NaHCO₃, and saturated brine, dried over anhydrous Na₂SO₄, and evaporated to give a crude product. The latter was purified repeatedly on silica gel using 15˜25% EtOAc in hexanes to give two isomeric product. The slow-eluting isomer was the title compound. ¹H NMR (CDCl₃, 500 MHz) [symbol] 7.57 (br s, INH), 7.35 (d, 8.5 Hz, 1H), 6.84 (d, 2.1 Hz, 1H), 6.77 (dd, 2.1 & 8.5 Hz, 1H), 3.86 (s, 3H), 2.67˜2.74 (m, 4H), 1.85˜1.95 (m, 4H). LC-MS: 3.60 min. (m/Z=202.2). The faster-eluting minor isomer was identified as 5-methoxy-2,3,4,9-tetrahydro-1H-carbazole. ¹H NMR (CDCl₃, 500 MHz) [symbol] 7.67 (br s, INH), 7.01 (dd, 8.0 & 7.1 Hz, 1H), 6.91 (d, 8.0 Hz, 1H), 6.48 (d, 7.6 Hz, 1H), 3.91 (s, 3H), 2.95˜2.98 (m, 2H), 2.70˜2.74 (m, 2H), 1.83˜1.93 (m, 4H). LC-MS: 3.63 min. (m/Z=202.2).

### Step B. 1-(7-methoxy-1,2,3,4-tetrahydro-9H-carbazol-9-yl)-3,3-dimethylbutan-2-one

To a solution of 33.7 mg 7-methoxy-2,3,4,9-tetrahydro-1H-carbazole from the Step A above in 1 mL anhydrous DMF was added 12 mg NaH (60% oil dispersion). After a few minutes, 31.3 mg of 1-bromo-3,3-dimethylbutan-2-one was added. The reaction mixture was purified on RP-HPLC using 60˜100% MeCN in water with 0.1% TFA to give the title compound as a solid following lyophilization. LC-MS: 4.02 min. (m/Z=300.2).

### EXAMPLE 2

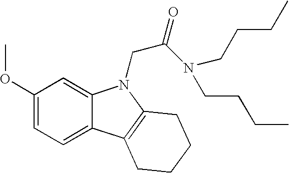

### N,N-Dibutyl-2-(7-methoxy-1,2,3,4-tetrahydro-9H-carbazol-9-yl)acetamide

### Step A. (7-Methoxy-1,2,3,4-tetrahydro-9H-carbazol-9-yl)acetic acid

To a solution of 0.25 g 7-methoxy-2,3,4,9-tetrahydro-1H-carbazole from the Step A Example 1 in 10 mL anhydrous DMF was added 150 mg NaH (60% oil dispersion). After 10 minutes, 0.21 g methyl bromoacetate was added and the resulting mixture stirred at room temperature for 5 hrs. Carefully add 1 mL each of water and 5 N NaOH to the reaction mixture. After stirring at room temperature over night, solvents were removed under reduced pressure. The residue was worked up using water and ether to give an acidic fraction containing the title compound. ¹H NMR (CDCl₃, 500 MHz) [symbol] 7.37 (d, 8.5 Hz, 1H), 6.79 (dd, 2.3 & 8.6 Hz, 1H), 6.70 (d, 2.3 Hz, 1H), 4.74 (s, 2H), 3.87 (s, 3H), 2.70˜2.72 (m, 2H), 2.63˜2.66 (m, 2H), 1.93˜1.98 (m, 2H), 1.84˜1.89 (m, 2H). LC-MS: 3.29 min. (m/Z=260.2).

### Step B. N,N-Dibutyl-2-(7-methoxy-1,2,3,4-tetrahydro-9H-carbazol-9-yl)acetamide

To a solution of 2.6 mg (7-methoxy-1,2,3,4-tetrahydro-9H-carbazol-9-yl)acetic acid from the Step A above in 0.5 mL anhydrous DMF were added 2.3 mg HOBt, 2.6 mg dibutyl amine, 7.7 mg EDC, and 2.6 mg DEA. After standing at room temperature over night, the reaction mixture was purified on RP-HPLC using 65˜100% MeCN gradient in water with 0.1% TFA. The title compound was obtained as a colorless solid after lyophilization. LC-MS: 4.31 min. (m/Z=371.3, 393.3).

### EXAMPLES 3˜17

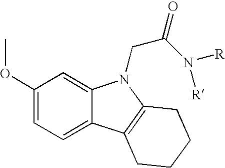

Examples 3˜17 in Table 1 were prepared from appropriate amine using the same procedure as described in Step B of Example 3. The preparation of the amines used for Examples 13˜16 have been described in WO/2004/04354 incorporated herein by reference in its entirety.

TABLE 1
Tetrahydrocarbazole Acetamides

| | | | LC-MS | |
|---|---|---|---|---|
| Example | R | R′ | tᵣ, min. | m/Z |
| 3 | n-Pr | n-Pr | 3.99 | 343.2, 365.3 |
| 4 | i-Amyl | i-Amyl | 4.55 | 399.3, 421.3 |
| 5 | i-Amyl | Et | 4.14 | 357.3, 379.3 |
| 6 | i-Bu | i-Bu | 4.24 | 371.3 |
| 7 | cyclopropylmethyl | n-Pr | 4.02 | 355.3 |
| 8 | cyclohexyl | Et | 4.17 | 369.4 |
| 9 | n-Bu | Et | 3.99 | 343.4 |
| 10 | n-Bu | n-Pr | 4.15 | 357.4 |
| 11 | 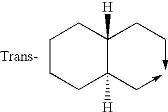 | | 4.23 | 381.4 |
| 12 | 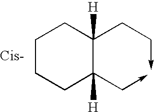 | | 4.17 | 381.4 |
| 13 | 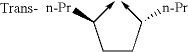 | | 4.43 | 397.4 |
| 14 | 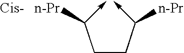 | | 4.47 | 397.4 |
| 15 | 3,3-Dimethylbutyl | Et | 4.25 | 371.4 |
| 16 | 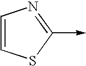 | Et | 3.89 | 370.3 |
| 17 | Neo-pentyl | Et | 4.10 | 357.4 |

### EXAMPLE 18

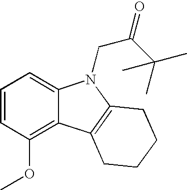

### 1-(5-Methoxy-1,2,3,4-tetrahydro-9H-carbazol-9-yl)-3,3-dimethylbutan-2-one

The title compound was prepared with 5-methoxy-2,3,4,9-tetrahydro-1H-carbazole from Example 1 Step A and using a similar procedure as described in Example 1 Step B. LC-MS: 4.07 min. (m/Z=300.2).

### EXAMPLES 19˜21

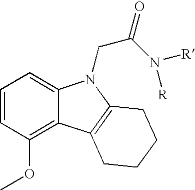

Examples 19˜21 in Table 2 were prepared starting with 5-methoxy-2,3,4,9-tetrahydro-1H-carbazole from Example 1 Step A and using similar procedures as described in Example 2.

TABLE 2
Isomeric Tetrahydrocarbazole Acetamides

LC-MS

| Example | R | R′ | tᵣ, min. | m/Z |
|---|---|---|---|---|
| 19 | n-Bu | n-Bu | 4.37 | 371.4, 393.3 |
| 20 | n-Pr | n-Pr | 4.04 | 343.2, 365.3 |
| 21 | i-Amyl | Et | 4.21 | 357.3, 379.3 |

### EXAMPLE 22

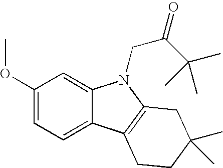

### 1-(7-Methoxy-2,2-dimethyl-1,2,3,4-tetrahydro-9H-carbazol-9-yl)-3,3-dimethylbutan-2-one

### Step A. 7-Methoxy-2,2-dimethyl-2,3,4,9-tetrahydro-1H-carbazole

The title compound was prepared using the procedure described in Step A Example 1 using 3,3-dimethylcyclohexanone and 3-methoxyphenylhydrazine hydrochloride. ¹H NMR (CDCl₃, 500 MHz) [symbol] 7.53 (br s, 1NH), 7.36 (br d, 1H), 6.84 (d, 2.0 Hz, 1H), 6.77 (dd, 2.0 & 8.5 Hz, 1H), 3.86 (s, 3H), 2.69 (br t, 2H), 2.49 (br s, 2H), 1.64 (t, 6.3 Hz, 2H), 1.07 (s, 6H). A faster-eluting minor isomer was also isolated from SGC. ¹H NMR (CDCl₃, 500 MHz) [symbol] 7.625 (br s, 1NH), 7.01 (dd, 8.0 & 8.0 Hz, 1H), 6.92 (d, 8.0 Hz, 1H), 6.49 (d, 7.8 Hz, 1H), 3.92 (s, 3H), 2.96 (br t, 5.6 Hz, 2H), 2.49 (s, 2H), 1.62 (t, 6.3 Hz, 2H), 1.07 (s, 6H). The latter was identified as 5-methoxy-2,2-dimethyl-2,3,4,9-tetrahydro-1H-carbazole.

### Step B. 1-(7-Methoxy-2,2-dimethyl-1,2,3,4-tetrahydro-9H-carbazol-9-yl)-3,3-dimethylbutan-2-one

The title compound was prepared using the procedure described in Step B Example 1 using 7-methoxy-2,2-dimethyl-2,3,4,9-tetrahydro-1H-carbazole and 1-bromo-3,3-dimethylbutan-2-one. LC-MS: 4.24 min. (m/Z=328.2, 350).

### EXAMPLE 23

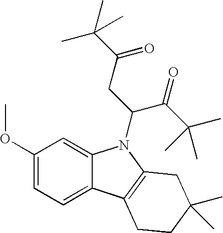

4-(7-methoxy-2,2-dimethyl-1,2,3,4-tetrahydro-9H-carbazol-9-yl)-2,2,7,7-tetramethyloctane-3,6-dione The title compound was isolated during the purification for Example 22. LC-MS: 4.90 min. (m/Z=448.3, 426.2).

### EXAMPLE 24

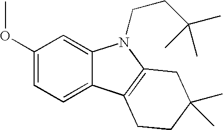

9-(3,3-Dimethylbutyl)-7-methoxy-2,2-dimethyl-2,3,4,9-tetrahydro-1H-carbazole The title compound was prepared by adding 3.6 mg 60% NaH oil dispersion to a solution of 17.2 mg 7-methoxy-2,2-dimethyl-2,3,4,9-tetrahydro-1H-carbazole from Step A Example 22 followed by 13.6 mg 1-bromo-3,3-dimethylbutane. After heating at 45° C. for 3 hrs, the reaction mixture was diluted with 1:1 dioxane and water and purified on RP-HPLC directly using 75˜100% MeCN gradient in water with 0.1% TFA to give the title compound as a colorless solid following lyophilization. LC-MS: 4.84 min. (m/Z=314.3).

### EXAMPLE 25

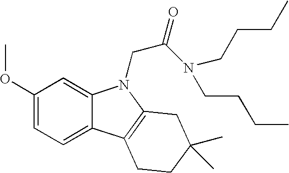

### N,N-Dibutyl-2-(7-methoxy-2,2-dimethyl-1,2,3,4-tetrahydro-9H-carbazol-9-yl)acetamide

### Step A. (7-Methoxy-2,2-dimethyl-1,2,3,4-tetrahydro-9H-carbazol-9-yl)acetic acid

The title compound was prepared from 7-methoxy-2,2-dimethyl-2,3,4,9-tetrahydro-1H-carbazole from Step A Example 22 using procedure described in Example 2 Step A. The crude product was further purified on RP-HPLC using 55˜100% MeCN gradient in water with 0.1% TFA to give pure title compound. ¹H NMR (CDCl₃, 500 MHz) [symbol] 7.38 (d, 8.4 Hz, 1H), 6.79 (dd, 2.3 & 8.5 Hz, 1H), 6.71 (d, 2.2 Hz, 1H), 4.73 (s, 2H), 3.87 (s, 3H), 2.70 (t, 6.3 Hz, 2H), 2.41 (s, 2H), 1.64 (t, 6.4 Hz, 2H), 1.08 (s, 6H). NOE difference spectrum from irradiating the 4.73 ppm signal gave positive NOE at 6.71 and 2.41 ppm. LC-MS: 3.55 min. (m/Z=288.2).

### Step B. N,N-Dibutyl-2-(7-methoxy-2,2-dimethyl-1,2,3,4-tetrahydro-9H-carbazol-9-yl)acetamide

(7-methoxy-2,2-dimethyl-1,2,3,4-tetrahydro-9H-carbazol-9-yl)acetic acid from Step A above, 11.5 mg HOBt, and 9.7 mg dibutylamine in 0 were added 24 mg EDC and 19.4 mg DEA. The reaction mixture was heated at 40° C. for 2 hrs and purified on RP-HPLC using 75˜100% MeCN gradient in water with 0.1% TFA. The title compound was obtained as a colorless solid following lyophilization. LC-MS: 4.46 min. (m/Z=399.3, 421.3).

### EXAMPLES 26˜40

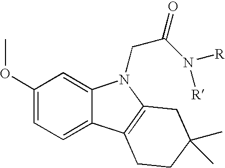

Examples 26˜40 in Table 3 were prepared from appropriate amine using the same procedure as described in Step B of Example 25. The preparation of the amines used for Examples 36˜39 have been described in WO2004/04354 incorporated herein by reference in its entirety.

TABLE 3
Dimethyltetrahydrocarbazole Acetamides

| | | | LC-MS | |
|---|---|---|---|---|
| Example | R | R′ | tᵣ, min. | m/Z |
| 26 | i-Bu | i-Bu | 4.41 | 399.3, 421.3 |
| 27 | cyclopropylmethyl | n-Pr | 4.22 | 383.3 |
| 28 | cyclohexyl | Et | 4.36 | 397.3 |
| 29 | n-Pr | n-Pr | 4.20 | 371.3 |
| 30 | n-Bu | Et | 4.21 | 371.3 |
| 31 | n-Bu | n-Pr | 4.34 | 385.3 |
| 32 | i-Amyl | Et | 4.33 | 385.3 |
| 33 | i-Amyl | i-Amyl | 4.68 | 427.4 |
| 34 | 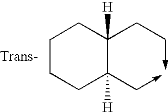 | | 4.41 | 409.4 |
| 35 | 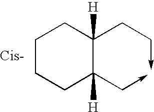 | | 4.37 | 409.4 |
| 36 | 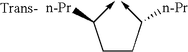 | | 4.58 | 425.4 |
| 37 | 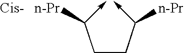 | | 4.61 | 425.4 |
| 38 | 3,3-Dimethylbutyl | Et | 4.43 | 399.4 |
| 39 | 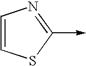 | Et | 4.43 | 398.3 |
| 40 | 3,3-Dimethylbutyl | n-Pr | 4.54 | 413.4 |

### EXAMPLE 41

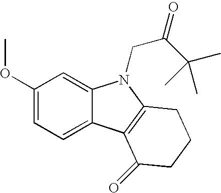

### 9-(3,3-Dimethyl-2-oxobutyl)-7-methoxy-1,2,3,9-tetrahydro-4H-carbazol-4-one

### Step A. 3-[(3-Methoxyphenyl)amino]cyclohex-2-en-1-one

A mixture of 25.62 g 3-methoxyaniline and 24.77 g cyclohexane-1,3-dione was heated at 130° C. under nitrogen for 6.5 hrs. The water formed was removed by distillation. The residue was dissolved in 300 mL chloroform and stirred with about 10 g activated charcoal for a few hrs, filtered, and evaporated to give the title compound. ¹H NMR (CDCl₃, 500 MHz) [symbol] 7.26 (t, 8.0 Hz, 1H), 6.72˜6.79 (m, 3H), 6.13 (br s, 1H), 5.65 (s, 1H), 3.81 (s, 3H), 2.52 (t, 6.3 Hz, 2H), 2.39 (t, 6.5 Hz, 2H), 2.04 (tt, 6.5 & 6.3 Hz, 2H). This crude product was used without further purification.

### Step B. 7-Methoxy-1,2,3,9-tetrahydro-4H-carbazol-4-one

3-[(3-methoxyphenyl)amino]cyclohex-2-en 1-one from Step A above in 1.5 L MeCN were added 6.60 g Pd(OAc)₂, and 80.10 g Cu(OAc)₂. This mixture was refluxed under nitrogen for 26 hrs. The hot mixture was filtered through 200 g silica gel with additional 2.5 L MeCN. The filtrate was evaporated to give a solid. This solid was boiled with 500 mL water, cooled to room temperature, and filtered. The solid was washed with water till the filtrate was no longer green. This crude product was purified using SGC using MeCN. The product from SGC was further washed with 1:1 EtOAc and MeCN to give the title compound as a brownish solid. ¹H NMR (CD₃OD, 500 MHz) [symbol] 7.87 (d, 8.4 Hz, 1H), 6.905 (d, 2.1 Hz, 1H), 6.82 (dd, 2.1 & 8.7 Hz, 1H), 3.82 (s, 3H), 2.98 (t, 6.2 Hz, 2H), 2.53 (t, 6.5 Hz, 2H), 2.22 (tt, 6.2 & 6.5 Hz, 2H). LC-MS: 2.32 min. (m/Z=216.1).

### Step C. 9-(3,3-Dimethyl-2-oxobutyl)-7-methoxy-1,2,3,9-tetrahydro-4H-carbazol-4-one

7-mMethoxy-1,2,3,9-tetrahydro-4H-carbazol-4-one from the Step B above in 1 mL anhydrous DMF were added 29.5 mg 1-bromo-3,3-dimethylbutan-2-one and 53.8 mg cesium carbonate. After 24 hrs at room temperature, the reaction mixture was diluted with 1:1 dioxane and water and purified on RP-HPLC using 50˜100% MeCN gradient in water with 0.1% TFA. The title compound was obtained as a colorless solid following lyophilization. LC-MS: 3.09 min. (m/Z=314.1).

### EXAMPLE 42

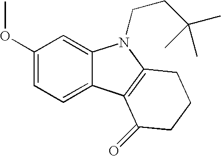

### 9-(3,3-Dimethylbutyl)-7-methoxy-1,2,3,9-tetrahydro-4H-carbazol-4-one

7-methoxy-1,2,3,9-tetrahydro-4H-carbazol-4-one from Example 41 Step B in 1 mL anhydrous DMF were added 27.2 mg 1-bromo-3,3-dimethylbutane and 53.8 mg cesium carbonate. The reaction mixture was heated at 45° C. for 1 hour and at 35° C. for 3 days. After cooling to room temperature, the reaction mixture was diluted with 1:1 dioxane and water and purified on RP-HPLC using 60˜100% MeCN gradient in water with 0.1% TFA. The title compound was obtained as a colorless solid following lyophilization. LC-MS: 3.66 min. (m/Z=300.2, 322.1).

### EXAMPLE 43

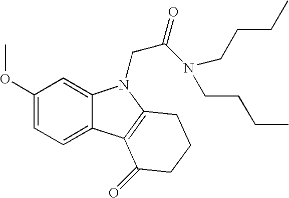

### N,N-Dibutyl-2-(7-methoxy-4-oxo-1,2,3,4-tetrahydro-9H-carbazol-9-yl)acetamide

### Step A. Ethyl (7-methoxy-4-oxo-1,2,3,4-tetrahydro-9H-carbazol-9-yl)acetate

To a solution of 1.822 g 7-methoxy-1,2,3,9-tetrahydro-4H-carbazol-4-one from Example 41 Step B in 40 mL anhydrous DMF were added 1.48 g ethyl bromoacetate and 2.897 g cesium carbonate. After stirring the mixture at room temperature for 3 days, it was diluted with 350 mL water and extracted with 4×100 mL EtOAc. The combined EtOAc extract was washed with water (3×150 mL) and saturated brine, dried over anhydrous Na₂SO₄, and evaporated to give the title compound as a yellow solid. It can be recrystallized from 30 mL EtOAc off white solid. ¹H NMR (CDCl₃, 500 MHz) [symbol] δ8.15 (d, 8.7 Hz, 1H), 6.94 (dd, 2.2 & 8.7 Hz, 1H), 6.73 (d, 2.1 Hz, 1H), 4.77 (s, 2H), 4.26 (q, 7.1 Hz, 2H), 3.88 (s, 3H), 2.90 (t, 6.2 Hz, 2H), 2.59˜2.62 (m, 2H), 2.26˜2.31 (m, 2H), 1.30 (t, 7.3 Hz, 3H). LC-MS: 2.85 min. (m/Z=302.1, 324.0).

### Step B. (7-Methoxy-4-oxo-1,2,3,4-tetrahydro-9H-carbazol-9-yl)acetic acid

A mixture of 1.17 g ethyl (7-methoxy-4-oxo-1,2,3,4-tetrahydro-9H-carbazol-9-yl)acetate from the Step A above in 50 mL MeOH, 4.15 mL water, and 0.85 mL 5 N NaOH was heated at 35° C. over night. The solvents were removed under reduced pressure. The residue was dissolved in water and extracted with 50 mL EtOAc. This extract was discarded. The aqueous layer was acidified with 1 mL concentrated HCl and extracted with 3×75 mL EtOAc. The combined was washed with saturated brine, dried over anhydrous Na₂SO₄, and evaporated to give the title compound as a yellowish solid. ¹H NMR (CD₃CN, 500 MHz) δ 9.79 (br s, 1OH), 7.94 (d, 8.5 Hz, 1H), 6.92 (d, 2.3 Hz, 1H), 6.86 (dd, 2.2 & 8.6 Hz, 1H), 4.91 (s, 2H), 3.83 (s, 3H), 2.87 (t, 6.2 Hz, 2H), 2.46˜2.49 (m, 2H), 2.17˜2.22 (m, 2H). LC-MS: 2.37 min. (m/Z=274.1).

### Step C. N,N-Dibutyl-2-(7-methoxy-4-oxo-1,2,3,4-tetrahydro-9H-carbazol-9-yl)acetamide

To a solution of 20.5 mg (7-methoxy-4-oxo-1,2,3,4-tetrahydro-9H-carbazol-9-yl)acetic acid from the Step B above in 0.9 mL anhydrous DMF were added 17.2 mg HOBt, 14.5 mg dibutyl amine, 28.8 mg EDC, and 29.1 mg DIEA. After standing at room temperature over night, the reaction mixture was purified on RP-HPLC using 50˜100% MeCN gradient in water with 0.1% TFA. The title compound was obtained as a colorless solid after lyophilization. LC-MS: 3.50 min. (m/Z=385.1).

### EXAMPLES 44˜58

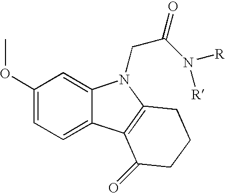

Examples 44˜58 in Table 4 were prepared from appropriate amine using the same procedure as described in Step C of Example 43. The preparation of the amines used for Examples 54˜57 have been described in WO2004/04354, incorporated herein by reference in its entirety.

TABLE 3
Oxotetrahydrocarbazole Acetamides

| | | | LC-MS | |
|---|---|---|---|---|
| Example | R | R′ | tᵣ, min. | m/Z |
| 44 | i-Bu | i-Bu | 3.43 | 385.1 |
| 45 | cyclopropylmethyl | n-Pr | 3.16 | 369.1 |
| 46 | cyclohexyl | Et | 3.32 | 383.1 |
| 47 | n-Pr | n-Pr | 3.12 | 357.1 |
| 48 | n-Bu | Et | 3.13 | 357.1 |
| 49 | n-Bu | n-Pr | 3.32 | 371.1 |
| 50 | i-Amyl | Et | 3.31 | 371.1 |
| 51 | i-Amyl | i-Amyl | 3.80 | 413.2 |
| 52 | 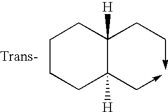 | | 3.40 | 395.1 |
| 53 | 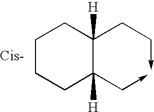 | | 3.33 | 395.1 |
| 54 | 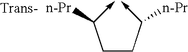 | | 3.64 | 411.1 |
| 55 | 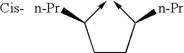 | | 3.68 | 411.1 |
| 56 | 3,3-Dimethylbutyl | Et | 3.45 | 385.1 |
| 57 | 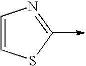 | Et | 2.99 | 384.0, 406.0 |
| 58 | 3,3-Dimethylbutyl | n-Pr | 3.62 | 399.1 |

A. Maxi-K Channel

The activity of the compounds can also be quantified by the following assay.

The identification of inhibitors of the Maxi-K channel is based on the ability of expressed Maxi-K channels to set cellular resting potential after transfection of both alpha and beta1 subunits of the channel in HEK-293 cells and after being incubated with potassium channel blockers that selectively eliminate the endogenous potassium conductances of HEK-293 cells. In the absence of maxi-K channel inhibitors, the transfected HEK-293 cells display a hyperpolarized membrane potential, negative inside, close to EK (−80 mV) which is a consequence of the activity of the maxi-K channel. Blockade of the Maxi-K channel by incubation with maxi-K channel blockers will cause cell depolarization. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)).

Oxanol is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, to a decrease in FRET. Thus, the ratio change (donor/acceptor) increases after membrane depolarization, which determines if a test compound actively blocks the maxi-K channel.

The HEK-293 cells were obtained from the American Type Culture Collection, 12301 Parklawn Drive, Rockville, Md., 20852 under accession number ATCC CRL-1573. Any restrictions relating to public access to the microorganism shall be irrevocably removed upon patent issuance.

Transfection of the alpha and beta1 subunits of the maxi-K channel in HEK-293 cells was carried out as follows: HEK-293 cells were plated in 100 mm tissue culture treated dishes at a density of 3×10⁶ cells per dish, and a total of five dishes were prepared. Cells were grown in a medium consisting of Dulbecco's Modified Eagle Medium (DMEM) supplemented with 10% Fetal Bovine serum, 1× L-Glutamine, and 1× Penicillin/Streptomycin, at 37° C., 10% CO₂. For transfection with Maxi-K hα(pCIneo) and Maxi-K hβ₁(pIRESpuro) DNAs, 150 μl FuGENE63 was added dropwise into 10 ml of serum free/phenol-red free DMEM and allowed to incubate at room temperature for 5 minutes. Then, the FuGENE63 solution was added dropwise to a DNA solution containing 25 μg of each plasmid DNA, and incubated at room temperature for 30 minutes. After the incubation period, 2 ml of the FuGENE63/DNA solution was added dropwise to each plate of cells and the cells were allowed to grow two days under the same conditions as described above. At the end of the second day, cells were put under selection media which consisted of DMEM supplemented with both 600 μg/ml G418 and 0.75 μg/ml puromycin. Cells were grown until separate colonies were formed. Five colonies were collected and transferred to a 6 well tissue culture treated dish. A total of 75 colonies were collected. Cells were allowed to grow until a confluent monolayer was obtained. Cells were then tested for the presence of maxi-K channel alpha and beta1 subunits using an assay that monitors binding of ¹²⁵I-iberiotoxin-D19Y/Y36F to the channel. Cells expressing ¹²⁵I-iberiotoxin-D19Y/Y36F binding activity were then evaluated in a functional assay that monitors the capability of maxi-K channels to control the membrane potential of transfected HEK-293 cells using fluorescence resonance energy transfer (FRET) ABS technology with a VIPR instrument. The colony giving the largest signal to noise ratio was subjected to limiting dilution. For this, cells were resuspended at approximately 5 cells/ml, and 200 μl were plated in individual wells in a 96 well tissue culture treated plate, to add ca. one cell per well. A total of two 96 well plates were made. When a confluent monolayer was formed, the cells were transferred to 6 well tissue culture treated plates. A total of 62 wells were transferred. When a confluent monolayer was obtained, cells were tested using the FRET-functional assay. Transfected cells giving the best signal to noise ratio were identified and used in subsequent functional assays.

For Functional Assays:

The transfected cells (2E+06 Cells/mL) are then plated on 96-well poly-D-lysine plates at a density of about 100,000 cells/well and incubated for about 16 to about 24 hours. The medium is aspirated of the cells and the cells washed one time with 100 μl of Dulbecco's phosphate buffered saline (D-PBS). One hundred microliters of about 9 μM coumarin (CC₂DMPE)-0.02% pluronic-127 in D-PBS per well is added and the wells are incubated in the dark for about 30 minutes. The cells are washed two times with 100 μl of Dulbecco's phosphate-buffered saline and 100 μl of about 4.5 μM of oxanol (DiSBAC₂(3)) in (mM) 140 NaCl, 0.1 KCl, 2 CaCl₂, 1 MgCl₂, 20 Hepes-NaOH, pH 7.4, 10 glucose is added. Three micromolar of an inhibitor of endogenous potassium conductance of HEK-293 cells is added. A maxi-K channel blocker is added (about 0.01 micromolar to about 10 micromolar) and the cells are incubated at room temperature in the dark for about 30 minutes.

The plates are loaded into a voltage/ion probe reader (VIPR) instrument, and the fluorescence emission of both CC₂DMPE and DiSBAC₂(3) are recorded for 10 sec. At this point, 100 μl of high-potassium solution (mM): 140 KCl, 2 CaCl₂, 1 MgCl₂, 20 Hepes-KOH, pH 7.4, 10 glucose are added and the fluorescence emission of both dyes recorded for an additional 10 sec. The ratio CC₂DMPE/DiSBAC₂(3), before addition of high-potassium solution equals 1. In the absence of maxi-K channel inhibitor, the ratio after addition of high-potassium solution varies between 1.65-2.0. When the Maxi-K channel has been completely inhibited by either a known standard or test compound, this ratio remains at 1. It is possible, therefore, to titrate the activity of a Maxi-K channel inhibitor by monitoring the concentration-dependent change in the fluorescence ratio.

The compounds of this invention were found to cause concentration-dependent inhibition of the fluorescence ratio with IC₅₀'s in the range of about 1 nM to about 20 μM, more preferably from about 10 nM to about 500 nM.

B. Electrophysiological Assays of Compound Effects on High-Conductance Calcium-Activated Potassium Channels

Methods:

Patch clamp recordings of currents flowing through large-conductance calcium-activated potassium (maxi-K) channels were made from membrane patches excised from CHO cells constitutively expressing the α-subunit of the maxi-K channel or HEK293 cells constitutively expressing both α- and β-subunits using conventional techniques (Hamill et al., 1981, Pflügers Archiv. 391, 85-100) at room temperature. Glass capillary tubing (Garner #7052 or Drummond custom borosilicate glass 1-014-1320) was pulled in two stages to yield micropipettes with tip diameters of approximately 1-2 microns. Pipettes were typically filled with solutions containing (mM): 150 KCl, 10 Hepes (4-(2-hydroxyethyl)-1-piperazine methanesulfonic acid), 1 Mg, 0.01 Ca, and adjusted to pH 7.20 with KOH. After forming a high resistance (>10⁹ ohms) seal between the plasma membrane and the pipette, the pipette was withdrawn from the cell, forming an excised inside-out membrane patch. The patch was excised into a bath solution containing (mM): 150 KCl, 10 Hepes, 5 EGTA (ethylene glycol bis(β-aminoethyl ether)-N,N,N′,N′-tetraacetic acid), sufficient Ca to yield a free Ca concentration of 1-5 μM, and the pH was adjusted to 7.2 with KOH. For example, 4.193 mM Ca was added to give a free concentration of 1 μM at 22° C. An EPC9 amplifier (HEKA Elektronic, Lambrect, Germany) was used to control the voltage and to measure the currents flowing across the membrane patch. The input to the headstage was connected to the pipette solution with a Ag/AgCl wire, and the amplifier ground was connected to the bath solution with a Ag/AgCl wire covered with a tube filled with agar dissolved in 0.2 M KCl. The identity of maxi-K currents was confirmed by the sensitivity of channel open probability to membrane potential and intracellular calcium concentration.

Data acquisition was controlled by PULSE software (HEKA Elektronic) and stored on the hard drive of a MacIntosh computer (Apple Computers) for later analysis using PULSEFIT (HEKA Elektronic) and Igor (Wavemetrics, Oswego, Oreg.) software.

Results:

The effects of the compounds of the present invention on Maxi-K channels was examined in excised inside-out membrane patches with constant superfusion of bath solution. The membrane potential was held at −80 mV and brief (100-200 ms) voltage steps to positive membrane potentials (typically +50 mV) were applied once per 15 seconds to transiently open Maxi-K channels. As a positive control in each experiment, maxi-K currents were eliminated at pulse potentials after the patch was transiently exposed to a low concentration of calcium (<10 nM) made by adding 1 mM EGTA to the standard bath solution with no added calcium. The fraction of channels blocked in each experiment was calculated from the reduction in peak current caused by application of the specified compound to the internal side of the membrane patch. Compound was applied until a steady state level of block was achieved. K, values for channel block were calculated by fitting the fractional block obtained at each compound concentration with a Hill equation. The K_{I} values for channel block by the compounds described in the present invention range from 0.01 nM to greater than 10 μM.

1-24. (canceled)

25. A compound which is: 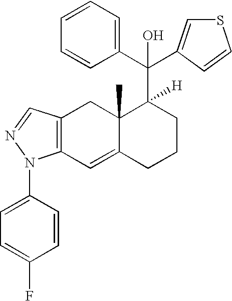
all possible diasteromers
or a pharmaceutically acceptable salt thereof.

26. A compound selected from the following group: 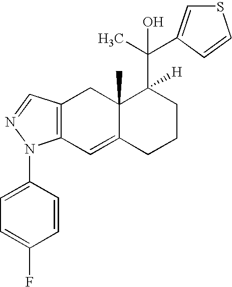
all possible diasteromers 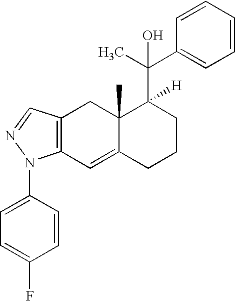
all possible diasteromers 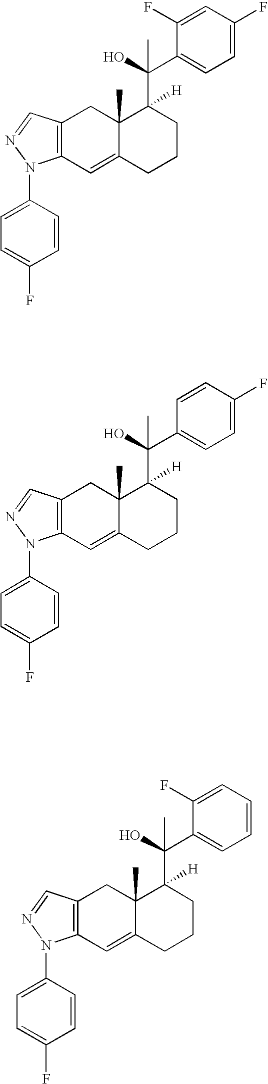
or a pharmaceutically acceptable salt of any of the foregoing compounds.
